SEQUENCE LISTING (10 sequences)

SEQ ID NO: 1 - DNA, 351 bp, Homo sapiens
  CDS (1)...(351)
atggattactacagaaaatatgcagctatctttctggtcacattgtcggtgtttctgcatgttctccattccgctcctgatgtgcaggattgcccagaatgcacgctacaggaaaacccattcttctcccagccgggtgccccaatacttcagtgcatgggctgctgcttctctagagcatatcccactccactaaggtccaagaagacgatgttggtccaaaagaacgtcacctcagagtccacttgctgtgtagctaaatcatataacagggtcacagtaatggggggtttcaaagtggagaaccacacggcgtgccactgcagtacttgttattatcacaaatcttaa

SEQ ID NO: 2 - protein, 116 aa, Homo sapiens
MDYYRKYAAIFLVTLSVFLHVLHSAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 4 - DNA, 417 bp, Homo sapiens
  CDS (1)...(417)
atgactgctctctttctgatgtccatgctttttggccttgcatgtgggcaagcgatgtctttttgtattccaactgagtatacaatgcacatcgaaaggagagagtgtgcttattgcctaaccatcaacaccaccatctgtgctggatattgtatgacacgggatatcaatggcaaactgtttcttcccaaatatgctctgtcccaggatgtttgcacatatagagacttcatctacaggactgtagaaataccaggatgcccactccatgttgctccctatttttcctatcctgttgctttaagctgtaagtgtggcaagtgcaatactgactatagtgactgcatacatgaagccatcaagacaaactactgtaccaaacctcagaagtcttatctggtaggattttctgtctaa

SEQ ID NO: 5 - protein, 138 aa, Homo sapiens
MTALFLMSMLFGLACGQAMSFCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 6 - protein, 118 aa, Homo sapiens
FCIPTEYTMHIERRECAYCLTINTTICAGYCMTRDINGKLFLPKYALSQDVCTYRDFIYRTVEIPGCPLHVAPYFSYPVALSCKCGKCNTDYSDCIHEAIKTNYCTKPQKSYLVGFSV

SEQ ID NO: 7 - DNA, 22 bp, Artificial Sequence
  Oligonucleotide primer
tcagaagaaaatcagaggaatc

SEQ ID NO: 8 - DNA, 23 bp, Artificial Sequence
  Oligonucleotide primer
gggacgttcagtagcggttgtag

SEQ ID NO: 9 - DNA, 20 bp, Artificial Sequence
  Oligonucleotide primer
ctgcccatggacaccgagac

SEQ ID NO: 10 - DNA, 23 bp, Artificial Sequence
  Oligonucleotide primer
ccgtttgcatatactcttctgag

(19) United States
(12) Patent Application Publication
(10) Pub. No.: US 2007/0010446 A1
(43) Pub. Date: Jan. 11, 2007

(54) METHODS FOR TREATING INFLAMMATION USING THYROID STIMULATING HORMONE

(75) Inventors: James D. Kelly, Mercer Island, WA (US); Shannon L. O'Hogan, Seattle, WA (US); Stacey R. Dillon, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(73) Assignee: ZymoGenetics, Inc.

(21) Appl. No.: 11/468,014

(22) Filed: Aug. 29, 2006

Related U.S. Application Data
(62) Division of application No. 11/005,591, filed on Dec. 6, 2004.
(60) Provisional application No. 60/527,344, filed on Dec. 5, 2003.

(51) Int. Cl. A61K 38/22

(52) U.S. Cl. 514/12

(57) ABSTRACT

Thyroid stimulating hormone has been shown to have anti-inflammatory activity. Polypeptides of thyroid stimulating hormone have a novel use as an anti-inflammatory agent as a stand-alone therapy, or in conjunction with other anti-inflammatory agents. In addition, thyroid stimulating hormone can be used to potentiate the anti-inflammatory activity of glucocorticoid treatment.

### CROSS REFERENCE TO RELATED APPLICATIONS

This application is a divisional application of U.S. patent application Ser. No. 11/005,591, filed Dec. 6, 2004, which claims the benefit of U.S. Provisional Application Ser. No. 60/527,344, filed Dec. 5, 2003, which applications are herein incorporated by reference in their entirity.

### BACKGROUND OF THE INVENTION

Inflammation normally is a localized, protective response to trauma or microbial invasion that destroys, dilutes, or walls-off the injurious agent and the injured tissue. Diseases characterized by inflammation are significant causes of morbidity and mortality in humans. While inflammation commonly occurs as a defensive response to invasion of the host by foreign material, it is also triggered by a response to mechanical trauma, toxins, and neoplasia. Excessive inflammation caused by abnormal recognition of host tissue as foreign, or prolongation of the inflammatory process, may lead to inflammatory diseases such as diabetes, asthma, atherosclerosis, cataracts, reperfusion injury, cancer, post-infectious syndromes such as in infectious meningitis, and rheumatic fever and rheumatic diseases such as systemic lupus erythematosus and rheumatoid arthritis. Thus, there is a need to produce agents that inhibit inflammation in many such diseases.

Glucocorticoids are used therapeutically as replacement therapy for individuals having adrenal insufficiencies, due to pathologies in the hypothalamus, anterior pituitary or the adrenal cortex. Glucocorticoids are also used for the treatment of a diverse number of non-endocrine diseases. Except in patients receiving replacement or substitution therapy, glucocorticoids are neither specific nor curative: they provide symptomatic relief by virtue of their anti-inflammatory and immunosuppressive properties. Glucocorticoids are used to treat rheumatic disorders such as rheumatoid arthritis, systemic lupus erythematosus, and a variety of vasculitic disorders such as polyarteritis nodosa, Wegener's granulomatosis and giant cell arteritis. In non-inflammatory degenerative joint diseases (e.g., osteoarthritis) or in a variety of regional pain syndromes (e.g., tendonitis or bursitis), glucocorticoids may be administered by local injection for the treatment of episodic disease flare-up.

Glucocorticoids are used to treat renal diseases, allergic disease including hay fever, serum sickness, urticaria, contact dermatitis, drug reactions, bee stings, allergic rhinitis and angioneurotic edema.

Glucocorticoids are also used to treat bronchial asthma, chronic obstructive pulmonary disease, chronic bronchitis and emphysema. Typically agents such as methylprednisolone or prednisone are used. Also inhaled glucocorticoids such as beclomethasone dipropionate, triamcinolone acetonide, flunisolide or budesonide can be used.

Glucocorticoids are used to treat a wide range of skin diseases including psoriasis, dermatitis, hidradenitis suppurativa, scabies, pityriasis rosea, lichen planus, and pityriasis rubra pilaris. Other inflammatory conditions in which glucocorticoids have been useful are toxic epidermal necrolysis, erythema multiforme, and sunburn.

Inflammatory bowel disease, ulcerative colitis and Crohn's disease can be treated with glucocorticoids. Glucocorticoids are also useful to treat chronic active hepatitis, alcoholic liver disease and severe hepatic disease. Glucocorticoids are used in the chemotherapy of acute lymphocytic leukemia and lymphomas because of their antilymphocytic effects. Glucocorticoids are also useful in the treatment of sarcoidosis, thrombocytopenia, autoimmune destruction of erythrocytes, organ transplantation, and in stroke and spinal cord injury.

However, as useful as glucocorticoids are, they do have severe side-effects. Two categories of toxic effects result from the therapeutic use of glucocorticoids: those resulting from withdrawal of glucocorticoid therapy and those resulting from continued use of supraphysiological doses. The most severe complication of the termination of glucocorticoid treatment is acute adrenal insufficiency, which results from too rapid a withdrawal of glucocorticoids after prolonged therapy, in which the hypothalamus/pituitary/adrenal (HPA) axis has been suppressed. Besides the consequences that result from the suppression of the HPA system, there are a number of other complications that result from prolonged glucocorticoid therapy, including fluid and electrolyte abnormalities, hypertension, hyperglycemia, increased susceptibility to infection, cataracts, growth arrest, fat redistribution, striae, ecchymosis, acne, hirsutism, and thymic atrophy.

Thus, there is a need to provide novel therapies to treat inflammation, including those administered in conjunction with glucocorticoid therapies that allow lower dosages of glucocorticoids to be used and thus lessen the side-effects of glucocorticoid treatment.

The present invention provides a novel use for Thyroid Stimulating Hormone, to treat inflammation and other uses that should be apparent to those skilled in the art from the teachings herein.

### SUMMARY OF THE INVENTION

Within an aspect of the invention is provided a method for treating inflammation, comprising administering a therapeutically sufficient amount of a TSH polypeptide to a mammal, wherein administration of the polypeptide results in a clinically significant improvement in the inflammatory condition of the mammal. Within an embodiment, the TSH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a) a decrease or inhibition in pain; b) a decrease or inhibition in swelling; c) a decrease or inhibition in redness; d) a decrease or inhibition in heat; e) and a decrease or inhibition in loss of function.

Within another aspect, the invention provides a method for treating inflammation, comprising administering a therapeutically sufficient amount of a TSH polypeptide to a mammal, wherein the inflammation is acute.

Within another aspect, the invention provides a method for treating inflammation, comprising administering a therapeutically sufficient amount of a TSH polypeptide to a mammal, wherein the inflammation is chronic.

Within another aspect, the invention provides a method for treating inflammation, comprising administering a therapeutically sufficient amount of a TSH polypeptide to a mammal, wherein the inflammatory condition of the mammal is associated with an autoimmune disease. Within an embodiment, the inflammation is associated with a rheumatic disorder. Within a further embodiment, the rheumatic disorder is rheumatoid arthritis, system lupus erythematosus, a vasculitic disorder, or another rheumatic disorder.

Within another aspect, the invention provides a method for treating inflammation, comprising administering a therapeutically sufficient amount of a TSH polypeptide to a mammal, wherein the inflammation is associated with an allergic response.

Within another aspect, the invention provides a method for treating inflammation, comprising administering a therapeutically sufficient amount of a TSH polypeptide to a mammal, wherein the inflammation is located in the respiratory tract. Within an embodiment, the inflammation is located in the lung, or sinus. Within another embodiment, the inflammation is associated with asthma, chronic obstructive pulmonary disease, chronic bronchitis, or emphysema.

Within another aspect, the invention provides a method for treating inflammation, comprising administering a therapeutically sufficient amount of a TSH polypeptide to a mammal, wherein the inflammation is located on the epidermis. Within an embodiment, the inflammation is associated with psoriasis, or dermatitis.

Within another aspect, the invention provides a method for treating inflammation, comprising administering a therapeutically sufficient amount of a TSH polypeptide to a mammal, wherein the inflammation is located in the gastrointestinal tract. Within an embodiment, the inflammation is associated with Inflammatory Bowel disease, ulcerative colitis, Crohn's disease, or inflammation associated diarrhea.

Within another aspect, the invention provides a method for treating inflammation, comprising administering a therapeutically sufficient amount of a TSH polypeptide to a mammal, wherein the inflammation is associated with Graft versus Host Disease. Within an embodiment, the inflammation is associated with single-organ or multi-organ failure.

Within another aspect, the invention provides a method for treating inflammation, comprising administering a therapeutically sufficient amount of a TSH polypeptide to a mammal, wherein the inflammation is associated with sepsis.

Within another aspect, the invention provides a method for treating inflammation, comprising administering a therapeutically sufficient amount of a TSH polypeptide to a mammal, wherein the inflammation is located in the liver. Within an embodiment, the inflammation is associated with chronic active hepatitis, alcoholic liver disease, or non-alcoholic fatty liver disease.

Within another aspect, the invention provides a method for treating inflammation, comprising administering a therapeutically sufficient amount of a TSH polypeptide to a mammal, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect, the invention provides a method for treating inflammation, comprising administering a therapeutically sufficient amount of a TSH polypeptide to a mammal, wherein treatment with the TSH polypeptide is used as an alternative to glucocorticoid treatment, and wherein administration of the polypeptide results in a clinically significant improvement in the inflammatory condition of the mammal.

Within another aspect, the invention provides a method for treating inflammation, comprising administering a therapeutically sufficient amount of a TSH polypeptide to a mammal, wherein treatment with the TSH polypeptide is used as an alternative to glucocorticoid treatment, and wherein administration of the polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

Within another aspect, the invention provides a method for reducing inflammation, comprising administering a therapeutically sufficient amount of a TSH polypeptide to a mammal, wherein administration of the polypeptide results in a clinically significant improvement in the inflammatory condition of the mammal. Within an embodiment, the TSH polypeptide forms a heterodimer, comprising the amino acid sequence as shown in SEQ ID NO:3, and the amino acid sequence as shown in SEQ ID NO:6. Within an embodiment, the clinically significant improvement in the inflammatory condition is selected from the group consisting of: a) a decrease or inhibition in pain; b) a decrease or inhibition in swelling; c) a decrease or inhibition in redness; a d) decrease or inhibition in heat; and e) a decrease or inhibition in loss of function.

Within another aspect, the invention provides a method for reducing inflammation, comprising administering a therapeutically sufficient amount of a TSH polypeptide to a mammal, wherein the inflammation is acute or chronic. Within an embodiment, the inflammation or inflammatory condition is associated with an autoimmune disease.

Within another aspect, the invention provides a method for reducing inflammation, comprising administering a therapeutically sufficient amount of a TSH polypeptide to a mammal, wherein the inflammation is located in the respiratory tract, on the epidermis, in the gastrointestinal tract, or the liver. Within an embodiment, the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, non-alcoholic fatty liver disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect, the invention provides a method for reducing inflammation, comprising administering a therapeutically sufficient amount of a TSH polypeptide to a mammal, wherein the TSH polypeptide is used as an alternative to glucocorticoid treatment.

Within another aspect, the invention provides a method for reducing inflammation, comprising administering a therapeutically sufficient amount of a TSH polypeptide to a mammal, wherein treatment with the TSH polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal. Within an embodiment, the TSH polypeptide forms a heterodimer, comprising the amino acid sequence as shown in SEQ ID NO:3, and the amino acid sequence as shown in SEQ ID NO:6. Within another embodiment, the clinically significant improvement in the inflammatory condition is selected from the group consisting of: a) a decrease or inhibition in pain; b) a decrease or inhibition in swelling; c) a decrease or inhibition in redness; d) a decrease or inhibition in heat; and e) a decrease or inhibition in loss of function.

Within another aspect, the invention provides a method for treating inflammation, comprising administering a therapeutically sufficient amount of a TSH polypeptide to a mammal in conjunction with one or more glucocorticoids, wherein the inflammation is acute.

Within another aspect, the invention provides a method for treating inflammation, comprising administering a therapeutically sufficient amount of a TSH polypeptide to a mammal in conjunction with one or more glucocorticoids, wherein the inflammation is chronic.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation or inflammatory condition is associated with an autoimmune disease. Within an embodiment, the inflammation is associated with a rheumatic disorder. Within further embodiment, the rheumatic disorder is rheumatoid arthritis, system lupus erythematosus, a vasculitic disorder, or another rheumatic disorder.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is associated with an allergic response. Within an embodiment, the inflammation is located in the respiratory tract. Within a further embodiment, the inflammation is associated with asthma, chronic obstructive pulmonary disease, chronic bronchitis, or emphysema.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is located on the epidermis. Within a further embodiment, the inflammation is associated with psoriasis, or dermatitis.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is located in the gastrointestinal tract. Within a further embodiment, the inflammation is associated with Inflammatory Bowel disease, ulcerative colitis, Crohn's disease, or inflammation associated diarrhea.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is associated with Graft versus Host Disease. Within an embodiment, the inflammation is associated with single-organ or multi-organ failure.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is associated with sepsis.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the inflammation is located in the liver. Within an embodiment, the inflammation is associated with chronic active hepatitis, alcoholic liver disease, or non-alcoholic fatty liver disease.

Within another aspect, the invention provides a method for treating inflammation, comprising administering a therapeutically sufficient amount of a TSH polypeptide to a mammal in conjunction with one or more glucocorticoids, wherein administration of the polypeptide results in a clinically significant improvement in the inflammatory condition of the mammal, TSH polypeptide forms a heterodimer, comprising the amino acid sequence as shown in SEQ ID NO:3, and the amino acid sequence as shown in SEQ ID NO:6, and wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

Within another aspect the invention provides a method for treating an inflammatory condition, comprising administering a therapeutically sufficient amount of a TSH polypeptide to a mammal in conjunction with one or more glucocorticoids, wherein treatment with the TSH polypeptide prevents or reduces a glucocorticoid-induced adverse side-effect. Within an embodiment, the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension. Within another embodiment, the level of glucocorticoid is reduced compared to treatment without the TSH polypeptide. Within another embodiment, the TSH polypeptide forms a heterodimer, comprising the amino acid sequence as shown in SEQ ID NO:3, and the amino acid sequence as shown in SEQ ID NO:6. Within another embodiment, the inflammatory condition is selected from the group consisting of: a) a decrease or inhibition in pain; b) a decrease or inhibition in swelling; c) a decrease or inhibition in redness; d) a decrease or inhibition in heat; and e) a decrease or inhibition in loss of function. Within another embodiment, the TSH polypeptide and the glucocorticoid are administered concurrently. Within another embodiment, the TSH polypeptide and the glucocorticoid are administered sequentially. Within another embodiment, the glucocorticoid is short-acting, intermdate-acting, or long-acting.

Within another aspect, the invention provides a method for treating inflammation or an inflammatory condition in a mammal, comprising administering a therapeutically sufficient amount of a TSH polypeptide to a mammal in conjunction with one or more glucocorticoids, wherein administration of the polypeptide results in a clinically significant improvement in the inflammation or inflammatory condition of the mammal, and wherein the glucocorticoid is selected from the group consisting of alclometasone dipropionate, amcinonide, beclomethasone dipropionate, betamethasone, betamethasone benzoate, betamethasone dipropionate, betamethasone sodium, betamethasone valerate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednislone, prednislone acetate, prednislone sodium phosphate, prednisolone tebutate, prednisone, triamcinolone, triamcinolone acetonide, triamcinolone diacetate and triamcinolone hexacetonide. Within an embodiment, the glucocorticoid is administered as a deriviative of alclometasone dipropionate, amcinonide, beclomethasone dipropionate, betamethasone, betamethasone benzoate, betamethasone dipropionate, betamethasone sodium, betamethasone valerate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednislone, prednislone acetate, prednislone sodium phosphate, prednisolone tebutate, prednisone, triamcinolone, triamcinolone acetonide, triamcinolone diacetate or triamcinolone hexacetonide.

Within another aspect, the invention provides a method for treating inflammation or an inflammatory condition in a mammal, comprising administering a therapeutically sufficient amount of a TSH polypeptide to a mammal in conjunction with one or more glucocorticoids, wherein administration of the polypeptide results in a decrease of a pro-inflammatory indicator. Within an embodiment, the pro-inflammatory indicator is measured by serum levels of pro-inflammatory cytokines or inflammation associated neutrophil infiltration. Within an embodiment, the pro-inflammatory cytokine is TNFα.

Within another aspect the invention provides a method for forming a peptide-receptor complex comprising, providing an immobilized receptor; and contacting the receptor with a peptide, wherein the peptide comprises the amino acid sequence as shown in SEQ ID NO:3 and the receptor is TSHR; whereby the receptor binds the peptide.

Within another aspect the invention provides a method for purifying TSH contained within a cell culture supernatant liquid comprising: applying the TSH-containing supernatant liquid to a chromatography column containing a cation exchange resin under conditions wherein the TSH binds to said cation exchange resin; eluting the TSH from the cation exchange resin and capturing a TSH-containing pool; applying the TSH-containing pool to a chromatography column containing a hydrophobic interaction resin under conditions wherein the TSH binds to said hydrophobic interaction resin; eluting the TSH from the hydrophobic interaction resin and capturing a TSH containing pool; applying the TSH-containing pool to a size-exclusion column and eluting the TSH from the size-exclusion resin and capturing the TSH in a TSH-containing pool.

### DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

Thyroid stimulating hormone (TSH), also called thyrotropin, is a glycoprotein hormone that is secreted from cells in the anterior pituitary called thyrotrophs and has been traditionally known to stimulate normal development and secretory activity of the thyroid gland. The novel methods of the present invention show that TSH can also be used to manipulate the immune response of an individual. The present invention thus comprises a method of administering TSH alone or in conjunction with a glucocorticoid.

The present invention provides methods for using TSH as an antiinflammatory agent in a broad spectrum of inflammatory conditions. In certain embodiments, the methods include using TSH to potentiate the effect of glucocorticoid treatment.

The methods of the present invention comprise administering a therapeutically effective amount of TSH, alone or in combination with other biologics or pharmaceuticals. The present invention provides methods of treating a mammal with chronic or acute inflammation, thereby reducing, ameliorating, limiting or preventing the inflammation. These methods are based on the discovery that TSH has anti-inflammatory activity in vivo.

As stated above, the methods of the present invention provide administering a therapeutically effective amount of TSH, alone or in combination with other biologics or pharmaceuticals. The present invention provides methods of treatment of a mammal with acute or chronic inflammation. Other aspects of the present invention provide methods for using TSH as an anti-inflammatory agent for inflammatory conditions, such as inflammatory conditions associated with autoimmune diseases, rheumatic disorders, allergic responses, Graft versus Host Disease, organ failure, sepsis, asthma, multiple sclerosis, inflammatory bowel disease, arthritis, critically ill patients, and immunosuppression.

In another aspect, the methods of the present invention also include a method of treating inflammation in a mammal comprising administering a therapeutically effective amount of TSH, alone or in combination with other biologics or pharmaceuticals, to replace or supplement treatment with glucocorticoids in inflammatory conditions, such as inflammatory conditions associated with autoimmune diseases, rheumatic disorders, allergic responses, Graft versus Host Disease, organ failure, sepsis, asthma, multiple sclerosis, inflammatory bowel disease, arthritis, critically ill patients, and immunosuppression.

### A. DESCRIPTION OF TSH POLYNUCLEOTIDES AND POLYPEPTIDES

Thyroid Stimulating Hormone (TSH) is a heterodimeric protein hormone comprised of an alpha subunit and a beta subunit that are non-covalently associated. The alpha subunit belongs to the glycoprotein hormones alpha chain family and has the polynucleotide and polypeptide amino acid sequences as shown in SEQ ID NOs:1 and 2, respectively. The signal sequence for the alpha subunit comprises amino acid residue 1 (Met) through amino acid residue 24 (Ser) of SEQ ID NO:2. The mature polypeptide for TSH alpha begins at amino acid residue 25 (Ala). The mature alpha subunit polypeptide is shown in SEQ ID NO:3. The beta subunit is unique to TSH and has the polynucleotide and polypeptide sequences as shown in SEQ ID NOs: 4 and 5, respectively. The signal sequence for the beta subunit comprises amino acid residue 1 (Met) through amino acid residue 20 (Ser) of SEQ ID NO:2. The mature polypeptide comprising the beta subunit of TSH begins at amino acid residue 21 (Phe). The mature beta subunit polypeptide is shown in SEQ ID NO:6. The TSH polypeptides are available from Genzyme (Thyrogen®, catalog number 36778; Genzyme Corporation, Cambridge, Mass.).

When a polynucleotide sequence encoding the mature polypeptide is expressed in a prokaryotic system, such as E. coli, the secretory signal sequence may not be required and the N-terminal Met will be present.

TSH exerts its effects through interaction with the thyroid-stimulating hormone (TSH), or thyrotropin, receptor. The TSH receptor (TSHR) is a member of the G-protein coupled, seven-transmembrane receptor superfamily. Activation of the TSH receptor leads to coupling with heterotrimeric G proteins, which evoke downstream cellular effects. The TSH receptor has been shown to interact with G proteins of subtypes Gs, Gq, G12, and Gi. In particular, interaction with Gs leads to activation of adenyl cyclase and increased levels of cAMP. See Laugwitz et al., Proc Natl Acad Sci USA 93: 116-20 (1996). Elevation of cAMP levels leads to activation of protein kinase A, a multi-potent protein kinase and transcription factor eliciting diverse cellular effects. See Bourne et al., Nature 349: 117-27 (1991).

The TSHR was originally identified in the thyroid as the principal activator of the thyroid gland, following exposure to the glycoprotein hormone, TSH. TSH release from the anterior pituitary stimulates the TSHR, resulting in secretion of thyroid hormone, stimulation of thyroid hormone synthesis, and cellular growth. TSH release is regulated by thyroid hormone levels. See, Utiger, in Endocrinology and Metabolism (Felig and Frohman, eds), pp. 261-347, McGraw-Hill, (2001).

Recently, the TSHR has been identified in many cell types not previously recognized, including cells of the immune system, brain, adipose, and reproductive organs. See, Example 2. These tissues are also targets of glucocorticoid action, suggesting a coordinate role for TSH and glucocorticoids as effectors of endocrine functions.

The TSH polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a TSH polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a TSH polypeptide into the secretory pathway of a host cell, a secretory signal sequence (also known as a leader sequence, prepro sequence or pre sequence) can be provided in the expression vector. The secretory signal sequence may be derived from another secreted protein (e.g., APO4, or t-PA) or synthesized de novo. The secretory signal sequence is operably linked to the TSH alpha or beta DNA sequence, i.e. SEQ ID NO:3 or SEQ ID NO:6, the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g., hygromycin resistance, multi-drug resistance, puromycin acetyltransferase) can also be used. Alternative markers that introduce an altered phenotype, such as green fluorescent protein, or cell surface proteins, such as CD4, CD8, Class I MHC, or placental alkaline phosphatase, may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant TSH baculovirus utilizes a transposon-based system described by Luckow (Luckow, V. A, et al., J Virol 67:4566-79, 1993). This system, which utilizes transfer vectors, is sold in the Bac-to-Bac kit (Life Technologies, Rockville, Md.). This system utilizes a transfer vector, pFastBac1 (Life Technologies) containing a Tn7 transposon to move the DNA encoding the TSH polypeptide into a baculovirus genome maintained in E. coli as a large plasmid called a “bacmid.” The pFastBac1 transfer vector utilizes the AcNPV polyhedrin promoter to drive the expression of the gene of interest, in this case TSH. However, pFastBac1 can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol Chem 270:1543-9, 1995. In such transfer vector constructs, a short or long version of the basic protein promoter can be used. Moreover, transfer vectors can be constructed which replace the native TSH secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native TSH secretory signal sequence. In addition, transfer vectors can include an in-frame fusion with DNA encoding an epitope tag at the C- or N-terminus of the expressed TSH polypeptide, for example, a Glu-Glu epitope tag (Grussenmeyer, T. et al., Proc. Natl. Acad. Sci. 82:7952-4, 1985). Using a technique known in the art, a transfer vector containing TSH is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses TSH is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO cell line (Invitrogen) derived from Trichoplusia ni (U.S. Pat. No. 5,300,435). Commercially available serum-free media are used to grow and maintain the cells. Suitable media are Sf900 II (Life Technologies) or ESF 921 (Expression Systems) for the Sf9 cells; and Ex-cellO405 (JRH Biosciences, Lenexa, Kans.) or Express FiveO (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the TSH polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant proteins is disclosed in U.S. Pat. Nos. 5,716,808, 5,736,383, 5,854,039, and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a TSH polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% BactoÔ Peptone (Difco Laboratories, Detroit, Mich.), 1% BactoÔ yeast extract (Difco Laboratories), 0.004% adenine and 0.006% L-leucine).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for TSH amino acid residues.

It is preferred to purify the polypeptides of the present invention to at least 80% purity, more preferably to at least 90% purity, even more preferably at least 95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant TSH proteins (including chimeric polypeptides and multimeric proteins) are purified by conventional protein purification methods, typically by a combination of chromatographic techniques. See, in general, Affinity Chromatography: Principles & Methods, Pharmacia LKB Biotechnology, Uppsala, Sweden, 1988; and Scopes, Protein Purification: Principles and Practice, Springer-Verlag, New York, 1994. Proteins comprising a polyhistidine affinity tag (typically about 6 histidine residues) are purified by affinity chromatography on a nickel chelate resin. See, for example, Houchuli et al., Bio/Technol. 6: 1321-1325, 1988. Proteins comprising a glu-glu tag can be purified by immunoaffinity chromatography according to conventional procedures. See, for example, Grussenmeyer et al., ibid. Maltose binding protein fusions are purified on an amylose column according to methods known in the art.

The polypeptides of the present invention can be isolated by a combination of procedures including, but not limited to, anion and cation exchange chromatography, size exclusion, and affinity chromatography. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39). Within additional embodiments of the invention, a fusion of the polypeptide of interest and an affinity tag (e.g., maltose-binding protein, an immunoglobulin domain) may be constructed to facilitate purification.

TSH polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989. In vitro synthesis is particularly advantageous for the preparation of smaller polypeptides.

Using methods known in the art, TSH proteins can be prepared as monomers or multimers; glycosylated or non-glycosylated; pegylated or non-pegylated; and may or may not include an initial methionine amino acid residue.

Conjugation of proteins with water-soluble polymers has been shown to enhance the circulating half-life of the protein, and to reduce the immunogenicity of the polypeptide (see, for example, Nieforth et al., Clin. Pharmacol. Ther. 59:636 (1996), and Monkarsh et al., Anal. Biochem. 247:434 (1997)). Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 40,000.

One example of a TSH conjugate comprises a TSH moiety, or mutant thereof, and a polyalkyl oxide moiety attached to the N-terminus of the TSH moiety. PEG is one suitable polyalkyl oxide. As an illustration, TSH can be modified with PEG, a process known as “PEGylation.” PEGylation of TSH can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). The methods of the present invention include administration of TSH and mutants thereof conjugated to water-soluble polymers, such as PEG.

Examples of biological activity for molecules used to identify mutants of TSH that are useful in the methods of the present invention include molecules that can bind to the TSHR with some specificity. Generally, a ligand binding to its cognate receptor is specific when the K_{D} falls within the range of 100 nM to 100 pM. Specific binding in the range of 100 mM to 10 nM K_{D} is low affinity binding. Specific binding in the range of 2.5 pM to 100 pM K_{D} is high affinity binding. In another example, biologically active TSH mutants are capable of some level of anti-inflammatory activity associated with wildtype TSH.

### B. USE OF TSH FOR INFLAMMATORY CONDITIONS

Inflammation has been traditionally characterized by pain, swelling, redness, heat and loss of function. Inflammatory diseases can result from chronic or acute events, such as, but not limited to trauma, injury, and stress, and autoimmune conditions, and can be the result of, for example, surgery, infection, allergy, or autoimmunity.

TSH can be used in treating inflammation that is associated with a variety of immune disorders. Such immune disorders comprise, for example, autoimmune disorders, rheumatic disorders, allergies, psoriasis, dermatitis, inflammatory intestinal disorders, Graft versus Host Disease, organ failure, and liver disease.

TSH receptors are found in many cells in the immune system, including targets of glucocorticoid action. These include monocyte/macrophages, T-cells, B-cells, dendritic cells, and polymorphonuclear leukocytes. See Example 2. Also see Bagriacik and Klein, J Immunol 164: 6158-65 (2000), and Kiss et al., Immunol Lett 55: 173-7 (1997). Flow cytometry using biotinylated TSH has been used to confirm expression of TSHR on the surface of these immune cell types (See Example 4). Also see Bagriacik and Klein, J Immunol 164: 6158-65, (2000). Activation of the TSHR has been shown to lead to increased cAMP in dendritic cells and T-cells, suggesting that these receptors are functional. Elevation of cAMP levels in a number of these cell types has been shown to inhibit the synthesis or secretion of several inflammatory cytokines, including IL-1, IL-6, IL-12, TNFα, and IFNg. See Delgado and Ganea, J Biol Chem 274: 31930-40 (1999). In addition, the production of inflammatory mediators such as nitric oxide is suppressed by elevated cAMP in macrophages. See Delgado et al., Ann NY Acad Sci 897: 401-14 (1999). These actions parallel the biochemical events described above for glucocorticoid action in the immune system.

Production of TNFα by immune cells is a significant component of inflammatory events. Glucocorticoids act to suppress TNFα through inhibition of NF-kB, as described above. Activation of the TSHR and elevation of cAMP also results in inhibition of TNFα expression by inhibition of phosphorylation of transcription factor c-Jun, which is phosphorylated by JNK kinase. See Delgado et al., J Biol Chem 273: 31427-36 (1998). C-Jun phosphorylation is required for high-affinity interaction with DNA sequences in the TNFα promoter region. In the absence of stimulus, these DNA sequences are occupied by the cAMP responsive element binding protein (CREB) transcription factor, which may act as a negative regulator of transcription. CREB transcription factor is activated by phosphorylation by protein kinase A downstream of elevated cAMP, which has been shown to exert negative regulation on TNFα expression. See Delgado et al., J Biol Chem 273: 31427-36 (1998). Thus, TSH can act to suppress TNFα, an important inflammatory mediator, alone or in adjunctive therapy with glucocorticoids.

Elevated cAMP downstream of TSH binding to immune cells represses transcription of IRF-1, an important component of the ets-2 transcription factor complex. Ets-2 is required for high-level expression of IL-12, an important stimulator of T cell mediated inflammatory responses. See Ma et al., J Biol Chem 272: 10389-95 (1997). IL-12 participates in T cell activation and cytotoxic lymphocyte functions and promotes the differentiation of T helper (TH) cells into the TH1 subset. See Trinchieri, Int Rev Immunol 16: 365-96 (1998). Glucocorticoids inhibit IL-12 synthesis primarily through inhibition of transcription factor NF-kB. Thus TSH can be used to decrease the inflammatory response in a mammal by administering TSH alone, or in conjunction with glucocorticoids. The effects of this administration can be measured by a decrease in IL-12. Methods for measuring IL-12 levels are commonly known to one skilled in the art, and are commercially available.

In thymocytes and T cells, cAMP elevation produces stabilization of IkBα and subsequent impairment of NF-kB nuclear translocation. See Manna and Aggarwal, J Immunol 161: 2873-80 (1998). Other studies have reported the inhibition of NF-kB transcriptional activity by elevated cAMP via competitive mechanisms. Competition for limiting amounts of coactivator CREB binding protein (CREBP) between phosphorylated CREB and NF-kB is suggested to result in lower levels of NF-kB transcriptional activity. See Ollivier et al., J Biol Chem 271: 20828-35 (1996).

IL-10 is an anti-inflammatory cytokine, which down-regulates IL-12 and TNFα production. Moderate exposure of peripheral blood lymphocytes to glucocorticoids increases IL-10 production. See Franchimont et al., J Clin Endocrinol Metab 84: 2834-9 (1999). IL-10 release is inhibited only at the highest concentrations of glucocorticoids. IL-10 synthesis is increased by elevated cAMP. See Platzer et al., Eur J Immunol 29: 3098-104 (1999). This suggests a therapeutic role for TSH, alone or in combination with glucocorticoids, in immune suppression by increasing synthesis of anti-inflammatory cytokines (IL-10) and by decreasing synthesis of proinflammatory cytokines (TNFα and IL-12).

The novel method of using TSH to treat inflammatory and immune disesases, as taught herein, can be used to target multiple components of the immune system. For example, as shown in Example 3, TSH treatment in vivo can suppress a delayed type hypersensitivity (DTH) reaction when administered either at the sensitization or at the challenge phase of the DTH response. The anti-inflammatory action of TSH is similar to that produced by the potent glucocorticoid dexamethasone in the DTH model of inflammation. Example 3 demonstrates the potent anti-inflammatory action of TSH administered alone, and suggests that co-administration of TSH with glucocorticoids would provide a means of decreasing glucocorticoid dosages. Another model to measure the anti-inflammatory effects of TSH is the LPS model described in Example 5.

In addition, the polypeptides of the present invention can be used in diagnosis of inflammatory diseases. Such diagnoses can be performed by means of a kit that provides for forming a peptide-receptor complex, wherein TSH is the peptide, and TSHR is the receptor, and wherein inflammation is detected by measuring a decrease in a proinflammatory indicator.

### C. USE OF TSH TO REPLACE OR SUPPLEMENT GLUCOCORTICOID THERAPY

Glucocorticoids can affect nearly all elements of inflammatory and immunologic responses. In general, glucocorticoids do not affect the condition or injury stimulating the primary response, but instead ameliorate the manifestations of the response to the initiating stressor. While it is generally accepted that basal glucocorticoid levels are permissive of the stress response and may enhance it, elevated levels act to limit the response, thus contributing to the recovery. See Sapolsky et al., Endocr Rev 21: 55-89 (2000). Such interplay may serve to modulate the magnitude and duration of immune responses and prevent the overproduction of cytokines that can threaten homeostasis.

Glucocorticoids exert their effects on responsive cells by binding to a classical steroid hormone receptor, which, upon the binding of hormone, translocates to the nucleus of the cell and causes altered rates of transcription of target genes. The glucocorticoid receptor (GR) is expressed throughout the body and is subject to very little feedback regulation. In inflammatory responses, glucocorticoids act to inhibit the production of acute-phase mediators of the immune response by repressing gene transcription. The most general effect of glucocorticoids is to inhibit the synthesis and release of cytokines and other inflammatory mediators that promote immune and inflammatory reactions. These include (but are not limited to) IL-1, IL-2, IL-3, IL-5, IL-6, IL-8, IL-12, TNFα (tumor necrosis factor alpha), IFNγ (interferon gamma), RANTES (regulated on activation, expressed and secreted by normal T cells), nitric oxide, eicosanoids, collagenase, plasminogen activator, histamine, and elastase. See Sapolsky et al., Endocr Rev 21: 55-89 (2000).

GR-mediated gene repression results from inhibition of nuclear factor NF-kB, a well-characterized component of the pro-inflammatory signaling pathway. See Rothwarf and Karin, Sci STKE 1999: RE1 (1999). The NF-kB complex is made up of a family of transcription factors related to the Rel protein. Following stimulation, the NF-kB complex is activated in the cytoplasm by phosphorylation and subsequent degradation of the inhibitory IkB subunit. The functional p65-p50 dimer is translocated to the nucleus and binds specific sequences in the regulatory region of NF-kB target genes. It is thought that GR-mediated inhibition of cytokine signaling through NF-kB accounts for the anti-inflammatory and immunosuppressive effects of glucocorticoids. See Miesfeld, in Endocrinology (DeGroot and Jameson, eds) Vol. 2, pp. 1647-1654, W. B. Saunders, (2001).

The relative potency of glucocorticoids in eliciting therapeutic responses correlates with receptor-binding activities, and duration of action in the systemic circulation. The commonly used glucocorticoids are classified as short-acting, intermediate-acting, and long-acting. Cortisol, the natural human glucocorticoid produced in the adrenal cortex, is a short-acting glucocorticoid. Other examples include cortisone, prednisone, prednisolone, and methylprednisolone. Triamcinolone is an example of an intermediate-acting glucocorticoid. Betamethasone and Dexamethasone are examples of long-acting glucocorticoids. See Axelrod, in Endocrinology (DeGroot and Jameson, eds) Vol. 2, pp. 1671-1682, W. B. Saunders, (2001). TSH will find use as a therapeutic for the replacement of, or in conjunction with glucocorticoid therapy in all clinical indications in which glucocorticoids are beneficial.

Glucocorticoids are among the most commonly used drugs. They are employed to treat many medical problems from minor skin conditions to life-threatening problems such as leukemia and organ transplant rejection. Clinical uses of TSH alone, or in conjunction with glucocorticoid therapy include but are not limited to allergic disease, such as asthma, drug reactions and urticaria. Included also are arthritis, especially rheumatoid arthritis. Other uses include inflammatory gastrointestinal disease, such as, for example, inflammatory bowel disease and ulcerative colitis, subacute hepatic necrosis, and regional enteritis. Autoimmune diseases such as lupus erythematosus, Crohn's disease, and autoimmune hemolytic anemia are also included as indications for treatment with TSH alone or in combination with glucocorticoids. TSH may be especially beneficial for the treatment of transplant rejection, including but not limited to, kidney, liver, heart, and lung transplant. TSH would be especially beneficial for treatment of blood dyscrasias such as leukemia, multiple myeloma, idiopathic thrombocytopenic purpura, and acquired hemolytic anemia. Other diseases that would benefit from TSH include sarcoidosis, eye diseases treated with glucocorticoids, neurologic disease, renal disease, and malignant hyperthermia. In addition TSH can also be used to treat sepsis and multi-organ failure.

### D. USE OF TSH TO REDUCE HPA SUPPRESSION

The hypothalamus, pituitary, and adrenal gland form a neuroendocrine circuit whose principal function is the regulation of cortisol production. Cortisol is the natural human glucocorticoid produced by the adrenal cortex. Cortisol exerts classical feedback regulation on this axis by decreasing the production of CRH (corticotrophin-releasing hormone) and ACTH (adrenocorticotrophic hormone). Feedback-regulation of ACTH secretion by elevated cortisol has been described as having fast, intermediate, and slow components. Both fast and intermediate feedback appears to be mediated by inhibition of the release of existing CRH and ACTH, rather than by inhibition of their synthesis. Fast feedback blunts the ACTH response to weaker stimuli, but allows response to strong stimuli such as endotoxin and surgery. As glucocorticoid concentrations increase, particularly with extended glucocorticoid therapy, slow feedback produces decreased or absent synthesis of ACTH, and eventually unresponsiveness of the pituitary to the administration of CRH. See Miller and Chrousos, in Endocrinology and Metabolism (Felig and Frohman, eds), pp. 387-524, McGraw-Hill, (2001).

During glucocorticoid therapy, adrenal cortex function can become significantly suppressed. Prolonged suppression can cause extreme atrophy of the adrenal cortex, due to privation of ACTH and its growth-stimulating activity. Recovery from such feedback suppression occurs slowly and is always an important consideration following the withdrawal of glucocorticoid therapy. Functional recovery of the adrenal cortex to the extent that a patient is able to mount an appropriate stress response following chronic glucocorticoid treatment takes from 1-12 months. As a result, glucocorticoid administration is typically continued at maintenance levels for several months following the end of therapeutic treatment. See Chrousos, in Endocrinology and Metabolism (Felig and Frohman, eds), pp. 609-632, McGraw-Hill (2001).

The time course of recovery of the adrenal cortex correlates with the total duration of previous glucocorticoid therapy as well as the total glucocorticoid dose. The rate of recovery is also determined by the doses given when the glucocorticoid is being tapered as well as the doses administered during the initial phases of the treatment. Recovery of the adrenal axis generally requires ACTH levels beyond the normal physiological range to reverse the atrophy associated with adrenal suppression. Elevated ACTH levels are typically seen for a period of months following the cessation of glucocorticoid therapy, before sufficient basal cortisol levels lower ACTH levels through feedback inhibition. However, even after ACTH levels return to the normal range, exposure of the adrenals to elevated ACTH following a significant stressor results in a blunted response. For this reason, low dose glucocorticoid administration is continued to ensure that patients can mount an adequate stress response. The use of ACTH to stimulate endogenous glucocorticoid production as a substitute for exogenous glucocorticoid administration has been extensively investigated. See Axelrod, in Textbook of Rheumatology (Kelley et al., eds), W B Saunders (1993). The use of ACTH dramatically reduces adrenal cortex suppression, and in some cases, results in an overactive and hyperplastic state. However, due to the concomitant mineralocorticoid and androgen stimulating properties of ACTH administration, ACTH is not a preferred treatment modality. The use of ACTH to reverse adrenal suppression following cessation of exogenous glucocorticoid treatment has also been studied. The administration of ACTH does not reverse the development or course of adrenal insufficiency. See Chrousos, in Endocrinology and Metabolism (Felig and Frohman, eds), pp. 609-632, McGraw-Hill (2001). To date, there is no method for hastening a return to normal HPA function once inhibition has resulted from glucocorticoid therapy.

Adrenal suppression is assessed clinically with standard practices. See Axelrod, in Textbook of Rheumatology (Kelley et al., eds) W B Saunders (1993). Glucocorticoids are withdrawn for approximately 24 hours, and a measured dose of ACTH is given. The relative increase in cortisol from baseline is measured at specific times following ACTH administration to assess the ability of the adrenals to respond adequately to a significant stress-related event. Due to fluctuations in basal cortisol levels, adrenal sufficiency is determined by increases in cortisol production, not by the absolute measured level.

TSH administration offers a novel method of preventing adrenal suppression. TSH acts upon the adrenal cortex through activation of TSH receptors in the adrenal cortex. This stimulates the production of cAMP in the cortex, which is necessary for the maintenance of normal cortical function. Example 1 demonstrates the cortical stimulation of TSH when a human adrenal cortex cell line, NCI-H295R, was used to study the signal transduction of TSH. TSH treatment produced a dose-dependent induction of cAMP comparable to forskolin, a constitutive inducer of adenyl cyclase.

Co-administration of TSH in glucocorticoid therapy can reduce or prevent adrenal atrophy, and in turn, adrenal suppression. This can be accomplished by two mechanisms. First, co-administration of TSH can allow the use of lower total doses of glucocorticoids, which can in turn result in less severe suppression, as described above. Second, the stimulatory effect of TSH on the adrenal cortex can ameliorate the atrophy of the cortex, preventing the long-term suppression of the adrenal gland, and restoring HPA axis response to significant stressors.

### E. FORMULATIONS AND ADMINISTRATION OF TSH

TSH can be administered in conjunction with or in place of glucocorticoid treatment. This means that TSH is administered before, during or after administration of the steroid, as well as a stand-alone therapy. Treatment dosages should be titrated to optimize safety and efficacy. Methods for administration include intravenous, peritoneal intramuscular, and topical. Pharmaceutically acceptable carriers include but are not limited to, water, saline, and buffers. Dosage ranges would ordinarily be expected from 0.1 μg to 100 μg per kilogram of body weight per day, with the exact dose determined by the clinician according to accepted standards, taking into account the nature and severity of the condition to be treated, patient traits, etc. Within this dosage range, a dose of 5 μg/kg/day can be used. Also within this range, a range from 5 μg/kg/day to 100 μg/kg/day can also be used. A useful dose to try initially would be 30 to 50 μg/kg per day. However, the doses may be higher or lower as can be determined by a medical doctor with ordinary skill in the art. For a complete discussion of drug formulations and dosage ranges see Remington's Pharmaceutical Sciences, 17th Ed., (Mack Publishing Co., Easton, Pa., 1990), and Goodman and Gilman's: The Pharmacological Bases of Therapeutics, 9th Ed. (Pergamon Press 1996).

For pharmaceutical use, the proteins of the present invention can be administered orally, rectally, parenterally, intracisternally, intravaginally, intraperitoneally, topically (as powders, ointments, drops or transdermal patch) bucally, or as a pulmonary or nasal inhalant. Intravenous administration will be by bolus injection or infusion over a typical period of one to several hours. In general, pharmaceutical formulations will include a TSH protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Doses of TSH polypeptide will generally be administered on a daily to weekly schedule. Determination of dose is within the level of ordinary skill in the art. The proteins may be administered for acute or chronic treatment, over several days to several months or years. In general, a therapeutically effective amount of TSH is an amount sufficient to produce a clinically significant change in an inflammatory condition.

While glucocorticoids are effective ubiquitous physiological regulators, glucocorticoid therapy can have severe adverse side effects. Therefore, it is highly desirable to lower the effective dosage of glucocorticoid in any therapeutic protocol. Adjunctive therapies that potentiate, or enhance, or even replace glucocorticoid action will allow the use of lower doses of glucocorticoids. The discovery of TSH as a mediator of immune suppression leads to the ability to administer TSH with or without glucocorticoids, or and thus produce equivalent therapeutic efficacy with lower glucocorticoid dosage.

TSH treatment may not lead to any measurable alterations in the lymphoid cell populations. Similar treatment with glucocorticoids leads to dramatic decreases in several lymphoid cell populations, particularly in the thymus, the site of T-lymphocyte maturation. As a result, treatment with glucocorticoids increases risk of infections, including bacterial, viral, fungal, and parasitic. See Chrousos, in Endocrinology and Metabolism (Felig and Frohman, eds), pp. 609-632, McGraw-Hill (2001). Co-administration of TSH to reduce glucocorticoid dosage or substitute therapy with TSH alone is expected to reduce the risk of these infections.

Some adverse consequences of glucocorticoid therapy are related more to the dose than to the duration of treatment. Avascular or ischemic necrosis of bone is a common side-effect of glucocorticoid therapy and is thought to be consequence of dosage. See Chrousos, in Endocrinology and Metabolism (Felig and Frohman, eds), pp. 609-632, McGraw-Hill (2001). There is evidence that steroid-induced cataracts, a significant risk in glucocorticoid treated patients, are due to high local concentrations of glucocorticoid in these individuals. The discovery of TSH as a modulator of inflammation permits glucocorticoid dosage to be reduced, with a concomitant reduction in side effects of glucocorticoid administration.

Other adverse consequences of glucocorticoid therapy may be ameliorated by TSH co-administration. Osteoporosis is a major limiting factor in long-term glucocorticoid therapy. Glucocorticoids are thought to act both on bone-forming cells, in part by producing apoptosis, and on osteoclasts, by stimulating bone resorption. High dosages of glucocorticoids are known to inhibit intestinal calcium absorption. See Singer, in Endocrinology and Metabolism (Felig and Frohman, eds), pp. 1179-1219, McGraw-Hill (2001). Reduction of glucocorticoid dosage has been investigated as a means of minimizing the adverse consequences in bone of long-term glucocorticoid use, and the use of the lowest possible therapeutic dose is strongly encouraged. See Chrousos, in Endocrinology and Metabolism (Felig and Frohman, eds), pp. 609-632, McGraw-Hill (2001). The presence of TSH receptors in osteoblasts (Example 2), the bone-forming cells, also suggests TSH is protective to glucocorticoid-mediated inhibition of bone formation. Thus, in bone, activation of the TSH receptor would increase cAMP, which stimulates differentiated functions in bone cells, thus inhibiting the pro-apoptotic effect of glucocorticoids. See Siddhanti and Quarles, J Cell Biochem 55: 310-20 (1994).

Additional side effects of glucocorticoid administration that would diminish with TSH co-administration include obesity, corticosteroid-induced psychosis, gastrointestinal hemorrhage, and benign intracranial hypertension.

Additionally, as indicated above, and taught herein, the anti-inflammatory effects of TSH will allow TSH administration to replace glucocorticoid therapy entirely.

TSH can also be used to treat arthritis as either stand-alone therapy, or in conjunction with a glucocorticoid, such as, for example, dexamethasone or prednisone. The effects of TSH can be measured in a in vivo model for collagen induced arthritis. See Tanaka, Y. et al., Inflamm. Res. 45:283-88, 1996.

The invention is further illustrated by the following non-limiting examples.

### F. EXAMPLES

### Example 1

TSH Activation of Adrenal Cortex Cells Results in cAMP Production

Summary: A human adrenal cortex cell line, NCI-H295R, was used to study signal transduction of TSH. NCI-H295R was transduced with recombinant adenovirus containing a reporter construct, a firefly luciferase gene under the control of cAMP response element (CRE) enhancer sequences. This assay system detects cAMP-mediated gene induction downstream of activation of Gs-coupled GPCR's (G-protein coupled receptors). Treatment of NCI-H295 with purified TSH heterodimer protein produced a dose-dependent induction of luciferase activity equal to or higher than that induced by 10 μM forskolin, a constitutive inducer of adenyl cyclase. Typically, TSH elicited a maximal response of 15-25-fold luciferase induction above control media. These results demonstrate TSH signaling through a GPCR in the adrenal cortex and the production of cAMP.

Experimental Procedure.

NCI-H295R cells were obtained from the ATCC(CRL-2128, Manassas, Va.) and cultured in growth medium as follows: 1:1 mixture of Dulbecco's modified Eagle's medium and Ham's F12 medium with L-glutamine (D-MEM/F-12; GIBCO, cat.# 11320-033) containing 25 mM HEPES buffer (GIBCO, Invitrogen, Carlsbad, Calif., cat.# 15630-080), 1 mM sodium pyruvate (GIBCO, cat.# 11360-070), 1% ITS+1 media supplement (Sigma St. Louis, Mo. cat# 12521) and 2.5% Nu-Serum I (BD Biosciences, Lexington, Ky. cat.#355100). Cells were cultured at 37° C. in a 5% CO₂ humidified incubator. One or two days before assaying, cells were seeded at 20,000 cells per well in a 96-well white opaque/clear bottom plate (BD Biosciences, cat.# 356650). One day before assay, cells were transduced with AV KZ55, an adenovirus vector containing KZ55, a CRE-driven luciferase reporter cassette, at 5000 particles per cell. Following overnight incubation, the cells were rinsed once with assay medium (D-MEM/F-12 supplemented with 0.1% bovine serum albumin, ICN Biomedicals, Inc., Aurora, Ohio, cat.# 103700), followed by incubation for four hours at 37° C. in assay medium to which test protein had been added. The plate was then washed with phosphate buffered saline (GIBCO, cat. # 20012-027).

Promega's Luciferase Assay System (Promega, Madison, Wis., cat. # E1500) was used to process the treated cells. Cell lysis buffer, 25 μl/well, was added to each well and incubated at room temperature for 15 minutes. Luciferase activity was measured on a microplate luminometer (PerkinElmer Life Sciences, Inc., model LB 96V2R) following automated injection of luciferase assay substrate.

### Example 2

Distribution of TSH Receptor Gene Expression.

RNA samples were surveyed for TSHR transcript using reverse transcriptase polymerase chain reaction (RT-PCR) amplification. Using standard procedures, RNA samples were isolated from tissues and cell lines, and RT-PCR was run with two separate pairs of primers. The first primer pair includes the forward primer (5′TCAGAAGAAAATCAGAGGAATC) (SEQ ID NO:7) and the reverse primer (5′GGGACGTTCAGTAGCGGTTGTAG) (SEQ ID NO:8), which amplify a 487 bp product. The amplified product spans an intron to control for signal arising from genomic DNA contamination. The second primer pair includes the forward primer (5′CTGCCCATGGACACCGAGAC) (SEQ ID NO:9) and the reverse primer (5′CCGTTTGCATATACTCTTCTGAG) (SEQ ID NO:10) and amplifies a 576 bp product. Additionally, TSHR expression was assessed from data in the published literature. Results are described below.

TSH Receptor in Immune Cells.

TSH-R is expressed in human CD14+ monocytes (decreasing expression after activation), in the human monocytic cell lines THP-1 and PMA-activated HL60 (but not in U937), in resting (but not activated) human NK cells, in human “resting” CD3+ (primarily CD4+) T cells, and in human B cells and B cell lines. Among mouse immune cell subsets, mTSH-R is expressed in CD4+ but not CD8+ T cells (decreasing with activation), in B cells (decreasing slightly with activation), and in an IFNγ-activated mouse macrophage line, J774.

Additionally, TSHR transcript has also been shown to be present in lymphocytes (Szkudlinski M. W., Fremont V., Ronin C., Weintraub B. D., (2002) Physiol Rev 82: 473-502) and other immune cell types (Bagriacik E U, and Klein J R, (2000) J Immunol 164: 6158-65).

TSH Receptor in Adrenal Gland.

RNA from the adrenal cortex carcinoma cell line H295R along with RNA isolated from several adult human normal adrenal glands were found to be positive for TSHR. Published literature also documents TSHR transcript in the adrenal gland (Dutton C. M., Joba W., Spitzweg C., Heufelder A. E., Bahn R. S., (1997) Thyroid 6: 879-84).

TSH Receptor in a Wide Variety of Cells and Tissue Types.

Extensive panels of RNAs were screened for TSHR and positive expression was found in thyroid, adrenal gland, kidney, brain, skeletal muscle, testis, liver, osteoblast, aortic smooth muscle, ovary, adipocytes, retina, salivary gland, and digestive tract. Similarly, the published literature documents TSHR expression in thyroid, kidney, thymus, adrenal gland, brain, retroocular fibroblasts, neuronal cells and astrocytes (Szkudlinski M. W., Fremont V., Ronin C., Weintraub B. D., (2002) Physiol Rev 82: 473-502 and Dutton C. M., Joba W., Spitzweg C., Heufelder A. E., Bahn R. S., (1997) Thyroid 6: 879-84).

### Example 3

Delayed Type Hypersensitivity in TSH-Treated Mice

Delayed Type Hypersensitivity (DTH) is a measure of T cell responses to specific antigen. In this response, mice are immunized with a specific protein in adjuvant (e.g., chicken ovalbumin, OVA) and then later challenged with the same antigen (without adjuvant) in the ear. Increase in ear thickness (measured with calipers) after the challenge is a measure of specific immune response to the antigen. DTH is a form of cell-mediated immunity that occurs in three distinct phases 1) the cognitive phase, in which T cells recognize foreign protein antigens presented on the surface of antigen presenting cells (APCs), 2) the activation/sensitization phase, in which T cells secrete cytokines (especially interferon-gamma; IFN-γ) and proliferate, and 3) the effector phase, which includes both inflammation (including infiltration of activated macrophages and neutrophils) and the ultimate resolution of the infection. This reaction is the primary defense mechanism against intracellular bacteria, and can be induced by soluble protein antigens or chemically reactive haptens. A classical DTH response occurs in individuals challenged with purified protein derivative (PPD) from Mycobacterium tuberculosis (TB), when those individuals injected have recovered from primary TB or have been vaccinated against TB. Induration, the hallmark of DTH, is detectable by about 18 hours after injection of antigen and is maximal by 24-48 hours. The lag in the onset of palpable induration is the reason for naming the response “delayed type.” In all species, DTH reactions are critically dependent on the presence of antigen-sensitized CD4+ (and, to a lesser extent, CD8+) T cells, which produce the principal initiating cytokine involved in DTH, IFN-γ.

In order to test for anti-inflammatory effects of TSH, a DTH experiment was conducted with three groups of C57BL/6 mice (Taconic, Germantown, N.Y.) treated with: I) PBS, II) 1.5 mg/kg Dexamethasone (Dex), and III) 2 mg/kg TSH. All of these treatments were given intraperitoneally two hours prior to the OVA re-challenge. The mice (8 per group) were first immunized in the back with 100 ug chicken ovalbumin (OVA) emulsified in Ribi in a total volume of 200 ul. Seven days later, the mice were re-challenged intradermally in the left ear with 10 ul PBS (control) or in the right ear with 10 ug OVA in PBS (no adjuvant) in a volume of 10 ul. Ear thickness of all mice was measured before injecting mice in the ear (0 measurement). Ear thickness was measured 24 hours after challenge. The difference in ear thickness between the 0 hour measurement and the 24 hour measurement is shown in Table 1. Control mice in the PBS treatment group developed a strong DTH reaction as shown by increase in the ear thickness at 24 hours post-challenge (Table 4, Expt #1). In contrast, mice treated with Dex or TSH had a lesser degree of ear thickness compared to controls. These differences were statistically significant, as determined by Student's t-test (Table 4, p values vs. PBS).

TABLE 1
TSH inhibits the Delayed Type Hypersensitivity (DTH) reaction when administered
either at the challenge or at the sensitization phase of the response.

| | | CHANGE IN EAR | | | |
|---|---|---|---|---|---|
| | | THICKNESS (×10⁻³ inch) | | | |
| EXPT | | TIME/ROUTE OF | LEFT EAR | RIGHT EAR | p value vs. |
| # | TREATMENT | TREATMENT | (PBS) | (OVA) | PBS |
| | PBS | Challenge (d 7) | 0.99 +/− 0.56 | 6.64 +/− 0.80 | — |
| 1 | 1.5 mg/kg Dex | i.p. | 0.23 +/− 0.77 | 2.89 +/− 1.29 | 0.000007 |
| (n = 8) | 2.0 mg/kg TSH | | 0.21 +/− 1.29 | 3.98 +/− 1.00 | 0.0001 |
| | PBS | Sensitization (d 0-4) | 1.50 +/− 0.53 | 7.78 +/− 1.70 | — |
| 2 | 1.5 mg/kg Dex | i.p. | 0.50 +/− 0.54 | 4.38 +/− 1.34 | 0.0014 |
| (n = 7) | 2.0 mg/kg TSH | | 1.26 +/− 0.99 | 6.07 +/− 0.67 | 0.029 |
| (ADX) | PBS | Challenge (d 7) | 0.33 +/− 0.67 | 6.16 +/− 1.54 | |
| 3 | 1.5 mg/kg Dex | i.p. | 0.07 +/− 0.61 | 3.71 +/− 0.64 | 0.0011 |
| (n = 7) | 2.0 mg/kg TSH | | 0.14 +/− 0.50 | 4.81 +/− 1.31 | 0.0967 |

A second DTH experiment was performed to evaluate anti-inflammatory effects of TSH when administered during the sensitization phase of the reaction (i.e. when T cells are responding to the antigen). Mice (7 per group) were administered PBS, Dex or TSH intraperitoneally once a day from days 0 to 4. The mice were then re-challenged with OVA or PBS on day 7 and ear thickness was measured on day 8. Again, both Dex and TSH significantly inhibited the DTH reaction (Table 1, Expt #2), suggesting that TSH can exert anti-inflammatory effects both early and late in the inflammation process.

Since the receptor for TSH is expressed by the adrenal glands, there was concern that the anti-inflammatory effects observed might be an indirect effect of increasing endogenous corticosteroid production in TSH-treated mice. A third DTH experiment was run to address this issue in adrenalectomized (ADX) mice. Once again, TSH and Dex-treated mice exhibited reduced ear swelling in response to the OVA re-challenge (Table 1, Expt #3). The results suggest that TSH activity is not mediated through endogenous glucocorticoid production by the adrenal glands.

Another approach to address the concern that the observed anti-inflammatory effects of TSH might be an indirect effect of increasing endogenous glucocorticoid production is to monitor thymus atrophy during treatment. One well-established side effect of increasing either exogenous or endogenous corticosteroid levels is substantial atrophy of the thymus, as the developing T cells are induced to undergo apoptosis. Therefore, the thymuses of the TSH and Dex-treated mice in the DTH experiments were analyzed. As shown in Table 2, while Dex-treated mice exhibited obvious thymic atrophy, neither the thymus weight nor the overall thymocyte cell counts were significantly affected by TSH treatment. Thymocytes were also analyzed by flow cytometry after staining the cells with fluorescently labeled antibodies to CD4, CD8 and CD3 (PharMingen, San Diego, Calif.), and it was found that the relative proportion of each thymocyte subset (CD4 single positive, CD8 single positive, CD4+CD8+ double positive, and CD4−CD8− double negative) in TSH-treated mice was not significantly different from that of the PBS-treated group. Thus, TSH seems to be mediating its anti-inflammatory effects in a manner distinct from that of endogenous glucocorticoids like Dex. This should prove to be an important benefit, as many of the adverse side effects of glucocorticoid treatment might potentially be avoided with TSH therapy.

TABLE 2
Unlike glucocorticoid treatment, TSH treatment in vivo does not induce thymic atrophy.
Thymuses were collected from mice in DTH expt #2 (in Table 4, above).

| TREATMENT | TIME OF | THYMUS | THYMOCYTE | | |
|---|---|---|---|---|---|
| GROUP | TREATMENT | WEIGHT | COUNT | p value vs. PBS | |
| n = 7 | IN DTH EXPT | (mg) | (×10⁻⁶ cells) | Weight | Counts |
| PBS | Sensitization | 61.9 +/− 12.4 | 140.3 +/− 36.6 | — | — |
| 1.5 mg/kg Dex | (days 0-4) | 30.1 +/− 7.6 | 46.0 +/− 7.4 | 0.000004 | 0.0023 |
| 2.0 mg/kg TSH | i.p. | 68.8 +/− 6.3 | 146.0 +/− 29.8 | 0.3619 | 0.8155 |

### Example 4

The TSH-R is Expressed on Human Monocytes.

U937, a human monoblastoid cell line, was obtained from ATCC (ATCC #CRL-1593.2) and maintained by serial passage in the media recommended by ATCC (RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 10%). To isolate human peripheral blood mononuclear cells (PBMC), whole blood (50 ml) was first collected from a healthy human donor and mixed 1:1 with PBS in 50 ml conical tubes. Thirty ml of diluted blood was then underlayed with 15 ml of Ficoll Paque Plus (Amersham Pharmacia Biotech, Uppsala, Sweden). These gradients were centrifuged 30 min at 500 g and allowed to stop without braking. The RBC-depleted cells at the interface (PBMC) were collected and washed 3 times with PBS.

Cells were resuspended in FACS Wash Buffer (WB=1×PBS/1% BSA/10 mM Hepes), counted in trypan blue, and 1×10⁶ viable cells of each type were aliquoted into wells of a 96-well round-bottomed plate. Cells were washed and pelleted, then incubated for 20 min on ice with 10 ug/ml of TSH-biotin. Cells were washed and then stained with 5 ug/ml streptavidin-PE (PharMingen) for an additional 20 min, to stain TSH-biotin-binding cells. Cells were washed thoroughly and pelleted, then resuspended in 0.4 ml of WB and analyzed on a FACSCalibur using CellQuest software (Becton Dickinson, Mountain View, Calif.).

As shown in Table 3 TSH-biotin clearly bound to the U937 monocyte line, as well as to primary human monocytes. These data partially agree with the expression pattern of TSH-R determined by RT-PCR (see Example 3) and by immunoprecipitation studies (see Bagriacik and Klein, J Immunol. 164: 6158-65, 2000), although one would expect to see TSH binding to the lymphoid cells as well. The absence of such staining in these experiments may reflect an underbiotinylation of the TSH, and/or low levels of surface expression of the TSH-R on lymphoid cells.

TABLE 3
TSH-biotin binds to a human monocyte cell line (U937),
as well as to monocytes in human peripheral blood. Mean
Fluorescence Intensities (MFI) are shown for TSH-biotin
(followed by streptavidin-phycoerythrin [SA-PE]) staining
of human PBMC, gated on lymphoid or myeloid cell subsets,
based on their light scatter characteristics (FSC vs. SSC).
TSH-biotin was used at 10 ug/ml. SA-PE was purchased from
Pharmingen and used at a 1:200 dilution of the stock. MFI
values reflecting a credible positive shift in the FL-2
channel are marked by an *.

| | | | | FL-2 MFI: |
|---|---|---|---|---|
| | | FL-2 MFI: | FL-2 MFI: | TSH-biotin + |
| Cell Source | Gated On: | unstained | 0 + SA-PE | SA-PE |
| U937 (hu | All live cells | 4.49 | 4.88 | 6.34* |
| mono line) | | | | |
| Hu PBMC, | Lymphoid cells | 2.13 | 2.50 | 2.63 |
| Donor 1 | | | | |
| Hu PBMC, | Myeloid cells | 3.43 | 4.44 | 7.78* |
| Donor 1 | | | | |
| Hu PBMC, | Lymphoid cells | 3.19 | 3.92 | 4.23 |
| Donor 2 | | | | |
| Hu PBMC, | Myeloid cells | 4.33 | 5.92 | 7.26* |
| Donor 2 | | | | |

### Example 5

LPS-Induced Mild Endotoxemia Mouse Model

The LPS-induced mild endotoxemia model is an in vivo model designed to mimic acute endotoxemia/sepsis by challenging mice with a low, non-lethal dose of bacterial endotoxin (lipopolysaccharide, LPS). Serum is collected at various timepoints (1-72 hours) after intraperitoneal LPS injection and analyzed for altered expression of a wide variety of pro- and anti-inflammatory cytokines and acute phase proteins that mediate the inflammatory response. The model provides a means to assess the potential anti-inflammatory effects of therapeutic candidates during a robust inflammatory response.

In this model, an initial experiment is run to determine the production of proinflammatory cytokines in response to an injection of LPS. For example, six-month old Balb/c (Charles River Laboratories, Wilmington, Mass.) female mice are injected with 25 μg LPS (Sigma) in sterile PBS intraperitoneally (i.p.). Serum samples are collected at 0, 1, 4, 8, 16, 24, 48 and 72 hours later, and are assayed for serum levels of inflammatory mediators, such as IL-1β, IL-6, TNFα, and IL-10. Commercially available ELISA kits (Biosource International, Camarillo, Calif.) can be used to deterime the serum levels. In this assay TNFα levels and IL-1β levels can be measured at 1 hour post-LPS injection, and IL-6, IL-10 and IL-10 can be measured at 4 hours post injection. From these inflammatory mediators, one or more is chosen as a representative biological marker(s) for the LPS model of mild endotoxemia.

Next, C57BL/6 mice (Charles River Laboratories; 5 mice/group) are treated i.p. with PBS, or the test compound in PBS 1 hour prior to LPS challenge. The mice are then challenged with 25 ug of LPS i.p. and are bled at 1 hour and 4 hours after LPS injection. The level of the representative biological marker(s) is analyzed by ELISA.

Suppression of the production of the pro-inflammatory cytokines (for example, TNFα) while enhancement of the expression of anti-inflammatory cytokines (for example, IL-6) will show the anti-inflammatory effects of the test compound.

When TSH was run as the test compound in this model, TNFα and IL-6 were chosen as the representative biological markers. TSH was administered at 2 mg/kg in PBS. Thyroxine (0.05 mg/kg) and Dexamethasone (0.015 mg/kg) were run as negative and positive controls, respectively. The anti-inflammatory effects of TSH in this model are shown in Table 4. Suppression of the production of the pro-inflammatory cytokines (TNFα), and enhancement of the expression of IL-6 (a cytokine that can have either pro- or anti-inflammatory properties) shows that TSH has anti-inflammatory effects in this model. This model can be optimized for dosage and choice of biological markers. The anti-inflammatory effects of TSH in this PLS-induced mild endotoxmia model are consistent with the anti-inflammatory effects seen in the DTH model.

TABLE 4
Anti-inflammatory Effects of TSH in the
LPS-induced Mild Endotoxemia Mouse Model

| | TNFα | IL-6 | | |
|---|---|---|---|---|
| Treatment | (1 hr) +/− S.E. | (4 hr) +/− S.E. | p values vs. PBS | |
| group | pg/ml | ng/ml | TNFα | IL-6 |
| PBS | 6990.8 +/− 1562.2 | 45.6 +/− 10.4 | | |
| Dex | 3798.4 +/− 415.7 | 35.2 +/− 2.8 | 0.033 | 0.26 |
| 0.015 mg/kg | | | | |
| TSH | 4345.5 +/− 385.2 | 73.1 +/− 11.2 | 0.139 | 0.11 |
| 2.0 mg/kg | | | | |
| Thyroxine | 4289.5 +/− 642.3 | 37.1 +/− 0.9 | 0.148 | 0.44 |
| (0.05 mg/kg) | | | | |

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method for treating or reducing inflammation, comprising administering a therapeutically sufficient amount of a TSH polypeptide to a mammal, wherein administration of the polypeptide results in a clinically significant improvement in the inflammatory condition of the mammal.

2. The method according to claim 1, wherein the TSH polypeptide forms a heterodimer, comprising the amino acid sequence as shown in SEQ ID NO:3, and the amino acid sequence as shown in SEQ ID NO:6.

3. The method according to claim 2, wherein the clinically significant improvement in the inflammatory condition is selected from the group consisting of:
a) a decrease or inhibition in pain;
b) a decrease or inhibition in swelling;
c) a decrease or inhibition in redness;
d) a decrease or inhibition in heat;
e) and a decrease or inhibition in loss of function.

4. The method according to claim 2, wherein the inflammation is acute or chronic.

5. The method according to claim 2, wherein the inflammation is associated with an autoimmune disease, an allergic response, asthma, chronic obstructive pulmonary disease, chronic bronchitis, emphysema, psoriasis, dermatitis, Inflammatory Bowel disease, ulcerative colitis, Crohn's disease, or inflammation associated diarrhea, Graft versus Host Disease, or sepsis.

6. The method according to claim 5, wherein the inflammation is associated with rheumatoid arthritis, system lupus erythematosus, a vasculitic disorder, or another rheumatic disorder, single-organ or multi-organ failure, chronic active hepatitis, alcoholic liver disease, or non-alcoholic fatty liver disease.

7. The method according to claim 2, wherein the inflammation is located in the respiratory tract, the lung, or sinus, on the epidermis, in the gastrointestinal tract, or in the liver.

8. The method according to claim 2, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

9. The method according to claim 2, wherein the treatment with the TSH polypeptide is used as an alternative to glucocorticoid treatment

10. The method according to claim 9, wherein the treatment with the TSH polypeptide prevents or reduces a glucocorticoid-induced adverse side-effect.

11. The method according to claim 10, wherein the glucocorticoid-induced adverse side-effect is selected from the group consisting of: adrenocortical suppression, osteoporosis, bone necrosis, steroid-induced cataracts, steroid-induced obesity, corticosteroid-induced psychosis, gastrointestinal hemorrhage, thymic atrophy, and benign intracranial hypertension.

12. The method according to claim 9, wherein the level of glucocorticoid is reduced compared to treatment without the TSH polypeptide

13. The method according to claim 9, wherein administration of the polypeptide results in a decrease of a pro-inflammatory indicator, and wherein the pro-inflammatory indicator is measured by serum levels of pro-inflammatory cytokines or inflammation associated neutrophil infiltration.

14. The method according to claim 9, wherein the inflammation is associated with an autoimmune disease, an allergic response, asthma, chronic obstructive pulmonary disease, chronic bronchitis, emphysema, psoriasis, dermatitis, Inflammatory Bowel disease, ulcerative colitis, Crohn's disease, or inflammation associated diarrhea, Graft versus Host Disease, or sepsis.

15. The method according to claim 14, wherein the inflammation is associated with rheumatoid arthritis, system lupus erythematosus, a vasculitic disorder, or another rheumatic disorder, single-organ or multi-organ failure, chronic active hepatitis, alcoholic liver disease, or non-alcoholic fatty liver disease.

16. The method according to claim 9, wherein the inflammation is located in the respiratory tract, the lung, or sinus, on the epidermis, in the gastrointestinal tract, or in the liver.

17. The method according to claim 9, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.

18. The method according to claim 2, wherein the TSH polypeptide is administered to the mammal in conjunction with one or more glucocorticoids.

19. The method according to claim 18, wherein the TSH polypeptide and the glucocorticoid are administered concurrently or sequentially.

20. The method according to claim 18, wherein the level of glucocorticoid is reduced compared to treatment without the TSH polypeptide.

21. The method according to claim 18, wherein the glucocorticoid is selected from the group consisting of alclometasone dipropionate, amcinonide, beclomethasone dipropionate, betamethasone, betamethasone benzoate, betamethasone dipropionate, betamethasone sodium, betamethasone valerate, clobetasol propionate, clocortolone pivalate, hydrocortisone, hydrocortisone acetate, hydrocortisone butyrate, hydrocortisone cypionate, hydrocortisone sodium phosphate, hydrocortisone sodium succinate, hydrocortisone valerate, cortisone acetate, desonide, desoximetasone, dexamethasone, dexamethasone acetate, dexamethasone sodium, diflorasone diacetate, fludrocortisone acetate, flunisolide, fluocinolone acetonide, fluocinonide, fluorometholone, flurandrenolide, halcinonide, medrysone, methylprednisolone, methylprednisolone acetate, methylprednisolone sodium, mometasone furoate, paramethasone acetate, prednislone, prednislone acetate, prednislone sodium phosphate, prednisolone tebutate, prednisone, triamcinolone, triamcinolone acetonide, triamcinolone diacetate and triamcinolone hexacetonide.

22. The method according to claim 18, wherein administration of the polypeptide results in a decrease of a pro-inflammatory indicator, and wherein the pro-inflammatory indicator is measured by serum levels of pro-inflammatory cytokines or inflammation associated neutrophil infiltration.

23. The method according to claim 18, wherein the inflammation is associated with an autoimmune disease, an allergic response, asthma, chronic obstructive pulmonary disease, chronic bronchitis, emphysema, psoriasis, dermatitis, Inflammatory Bowel disease, ulcerative colitis, Crohn's disease, or inflammation associated diarrhea, Graft versus Host Disease, or sepsis.

24. The method according to claim 18, wherein the inflammation is associated with rheumatoid arthritis, system lupus erythematosus, a vasculitic disorder, or another rheumatic disorder, single-organ or multi-organ failure, chronic active hepatitis, alcoholic liver disease, or non-alcoholic fatty liver disease.

25. The method according to claim 18, wherein the inflammation is located in the respiratory tract, the lung, or sinus, on the epidermis, in the gastrointestinal tract, or in the liver.

26. The method according to claim 18, wherein the mammal has a disease selected from the group consisting of: rheumatoid arthritis, systemic lupus erythematosus, polyarteritis nodosa, Wegener's granulomatosis, giant cell arteritis, renal disease, allergic disease, asthma, chronic obstructive pulmonary disease, chronic bronchitis, emphysema, psoriasis, inflammatory bowel disease, ulcerative colitis, Crohn's disease, chronic active hepatitis, alcoholic liver disease, hepatic disease, acute lymphocytic leukemia, lymphomas, sarcoidosis, thrombocytopenia, autoimmune hemolytic anemia, organ transplantation, stroke, spinal cord injury, drug reactions, urticaria, subacute hepatic necrosis, multiple myeloma, idiopathic thrombocytopenic purpura, acquired hemolytic anemia and malignant hyperthermia.
